(19) United States
(12) Patent Application Publication
(10) Pub. No.: US 2007/0020203 A1
(43) Pub. Date: Jan. 25, 2007

(54) Skin care composition

(76) Inventors: Ratan K. Chaudhuri, Lincoln Park, NJ (US); Zoia Lascu, Valhalla, NY (US); Germain Puccetti, Yorktown Heights, NY (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/481,259

(22) Filed: Jul. 6, 2006

Related U.S. Application Data
(60) Provisional application No. 60/696,788, filed on Jul. 7, 2005.

(51) Int. Cl. A61K 8/97; A61K 36/48; A61K 36/185

(52) U.S. Cl. 424/59; 424/74; 424/725; 424/757

(57) ABSTRACT

Skin care compositions comprising certain ratios of phyllanthus Emblica extracts, licorice extracts and niacinamide. Certain embodiments provide a synergistic response to lightening/even-toning the skin. These skin care compositions comprise skin lightening/even-toning formulations, sunscreen compositions, compositions for photoprotecting skin or reducing a stretch mark or compositions for repairing damaged skin.

This application claims the benefit of the filing date of U.S. Provisional Application Ser. No. 60/696,788 filed Jul. 7, 2005 the entire disclosure of which is incorporated by reference herein.

### FIELD OF THE INVENTION

The present invention is directed to a skin care composition comprising low molecular weight hydrolysable tannins obtained from phyllanthus emblica extracts, (as exemplified by a proprietary trademark Emblica), together with licorice extracts containing about 20% glabridin, and with niacinamide. These compositions are especially effective for skin lightening/even-toning and can also be used in compositions for protection against the sun (sun screens and the like) and for regulating the appearance of skin, and an anti-aging composition.

### BACKGROUND OF THE INVENTION

The pigmentation of the skin, due to synthesis and dispersion of melanin in the epidermis, is of great cosmetic and societal significance. The pigmentation of the skin is also the key physiological defense against sun-induced damage, such as sunburn, photoaging and photocarcinogenesis. Skin care products with skin lightening/even-toning benefits are known. Publications which describe skin care products include:
U.S. Pat. No. 6,609,875, entitled “Plate material carrying apparatus” and issued to Araki,
U.S. Pat. No. 6,649,150, entitled “Skin-lightening” and issued to Chaudhuri et al.,
U.S. Pat. No. 5,693,327, entitled “Herbal compositions” and issued to Shah,
US Published Application No: 2003/194452, entitled “Improved Herbal Composition Having Antiallergic Properties and a Process for the Preparation Thereof” and filed by Agarwal et al.,
US Published Application No: 2004/0028642 A1, entitled “Cosmetic composition comprising an extract of emblica officinalis and methods of using same” and filed by Hansenne et al.,
US Published Application No: 2004/164435, entitled “Methods and apparatus for producing granular compositions” and filed by Pinyayev et al.,
US Published Application No: 2004/156873, entitled “Topically Bioavailable Acne and Rosacea Treatment Compositions” and filed by Gupta,
US Published Application No: 2004/219124, entitled “Cosmetic and Pharmaceutical Masks Based on Ion-Pair Delivery System” and filed by Gupta et al.,
US Published Application No: 2004/0086560, entitled “Skin-lightening” and filed by Chaudhuri et al.,
US Published Application No: 2004/0076699, entitled “Topical anhydrous delivery system” and filed by Chaudhuri et al.,
US Published Application No: 2004/0126446, entitled “Enriched aqueous components of emblica officinalis” and filed by Chaudhuri et al.,
US Published Application No: 2004/0166069 A1, entitled “Boosting Tyrosinase Inhibiting Activity of Skin Whitening and Sunscreen Compositions” and filed by Gupta,
US Published Application No: 2004/0253332 A1, entitled “Method for regulating the appearance of skin containing combination of skin care actives” and filed by Chaudhuri,
US Published Application No: 2004/0208902 A1, entitled “Controlled-release nano-diffusion delivery systems for cosmetic and pharmaceutical compositions” and filed by Gupta,
US Published Application No: 2004/0146539, entitled “Topical Nutraceutical Compositions with Selective Body Slimmning and Tone Firming Antiaging Benefits” and filed by Gupta,
US Published Application No: 2003/0157202, entitled “Lightening compositions and methods of use” and filed by Mahalingam et al.,
US Published Application No: 2004/0156798, entitled “Cosmetic compositions containing Rooibus tea extract” and filed by Reinhart et al.,
US Published Application No: 2004/0126344, entitled “Compositions having glycolipid to lighten skin and alter post-inflammatory hyperpigmentation” and filed by Mahalingam et al.,
US Published Application No: 2002/0141955, entitled “Method for slowing the decomposition of a cosmetic composition” and filed by Zimmerman et al.,
ISBN 1416002448, “Procedures in Cosmetic Dermatology Series,” Part 2, Cosmeceutical Actives, Chap. 16, Skin Lightening Agents, Marta I. Rendon and Jorge I. Gaviria,
Published International Application No. WO 03/059313 A1, entitled “Skin-Lightening Composition” and filed by Chaudhuri et al.,
Published International Application No. WO 04/041234 A1, entitled “Topical Anhydrous Delivery System For Antioxidants” and filed by Chaudhuri et al.,
Published International Application No. WO 04/041231 A1, entitled “Method For Protection Of Skin Against Sun-Induced Damage By Oral Administration Of An Extract Of Emblica Officinalis (syn. Phyllanthus Emblica)” and filed by Chaudhuri,
Published International Application No. WO 04/062635 A1, entitled “Compositions For Skin Lightening Comprising Bis-Pantoyl-Cystamine” and filed by Burdick et al.,
U.S. Ser. No. 10/691,959, Publication No. 2005-0089590 A1, published Apr. 28, 2005,
U.S. Ser. No. 10/616,299, Publication No. 2005-0008590 A1, published Jan. 13, 2005,
U.S. Ser. No. 10/803,160, Publication No. 2004-0253332 A1, published Dec. 16, 2004,

Skin care products combining both ant-aging and skin lightening/even-toning benefits in a single formulation are desired. In addition, more efficacious skin lightening/even toning products are desired.

### SUMMARY OF THE INVENTION

In one aspect of the present invention a skin care composition is provided comprising a combination of an extract of Phyllanthus emblica, a licorice extract, and niacinamide in prescribed proportions. Preferably, the composition also includes a pharmaceutically or cosmetically acceptable carrier(s). The skin care composition can be in the form of a skin lightening/even-toning composition, a sunscreen composition, a composition for photoprotecting skin or a composition for repairing photo-damaged skin.

The prescribed ratios of the three active components with respect to one another are as follows:
the ratio of the extract of Phyllanthus emblica to the extract of licorice is from 2 to 0.01, preferably 1 to 0.05 and even more preferably 1 to 0.1;
the ratio of phyllanthus emblica to niacinamide is from 0.5 to 10, preferably 1 to 8, and even more preferably 1 to 5; and
the ratio of the extract of licorice to the niacinamide is from 0.01 to 10, preferably 0.05 to 5, and even more preferably 0.1 to 5. All of the ratios above are on a weight basis of dry solids.

In preferred embodiments, the prescribed ratios of the three active components with respect to one another are as follows:
the ratio of the extract of Phyllanthus emblica to the extract of licorice is from 1 to 0.05 and even more preferably 1 to 0.1;
the ratio of phyllanthus emblica to niacinamide is from 1 to 8, and even more preferably 1 to 5; and
the ratio of the extract of licorice to the niacinamide is from 0.05 to 5, and more preferably 0.1 to 5; all on a weight basis of dry solids.

For the even more preferred embodiments, the prescribed ratios of the three active components with respect to one another are as follows:
the ratio of the extract of Phyllanthus emblica to the extract of licorice is from 1 to 0.1;
the ratio of phyllanthus emblica to niacinamide is from 1 to 5; and
the ratio of the extract of licorice to the niacinamide is 0.1 to 5, all on a weight basis of dry solids.

In another aspect of the present invention, a skin care composition is provided comprising a combination of an extract of Phyllanthus emblica, a licorice extract, and niacinamide in proportions which provide a synergistic response in changing skin tone as compared to an equal amount of any of the individual components, on a weight basis. Preferably, the composition includes a pharmaceutically or cosmetically acceptable carrier(s).

In a further aspect of this invention, a skin composition is provided comprising a combination of Phyllanthus Emblica, licorice extract and niacinamide in proportions which provide a synergistic response in reducing stretch marks, repairing photodamaged skin or photoprotecting skin as compared to an equal amount of any of the individual components on a weight basis.

The skin care composition can be in the form of a skin lightening/even-toning composition, a sunscreen composition, a composition for photoprotecting skin or a composition for reducing the appearance of stretch marks, or a composition for repairing photo-damaged skin.

A synergistic response in changing skin tone includes
a response which provides a lighter skin tone for one or more skin types,
a response which provides a greater change in skin tone for one or more skin types,
a response which provides a greater change in skin tone over a short treatment period of less than three weeks for one or more skin types,
a response that provides more uniform skin color for one or more skin types,
a response that provides more uniform skin color over a short treatment period of less than three weeks for one or more skin types, or
a response with one or more of the above characteristics.

A synergistic response in reducing the appearance of stretch marks includes:
a response which provides a change in the color of a stretch mark which more closely resembles the surrounding skin; or
a response which provides a change in the color of a stretch mark to resemble the surrounding skin over a short treatment period of less than three weeks.

A synergistic response with respect to repairing photodamage skin includes:
a response which provides a change in the color of photodamaged skin to more closely resemble the color of undamaged skin; or
a response which provides a change in the color of photodamaged skin to resemble the color of undamaged skin in a short treatment period of less than three weeks.

A synergistic response with respect to photoprotecting skin includes a response which provides a reduction in permanent skin discoloration for a given exposure to sunlight or simulated sunlight.

Phyllanthus Emblica is a plant commonly found in India, China, Pakistan, Nepal and other countries. The extracts of Phyllanthus Emblica employed in this invention are derived from the fruit of this plant. Fruits of Phyllanthus Emblica obtained from any geographical location are suitable for preparing the extracts used in this invention and any extract from these fruits containing at least the following low molecular weight tannins is suitable for use in this invention.
Emblicanin A /Emblicanin B (gallic/ellagic acid derivatives of 2-keto-glucono-.delta.-lactone);
Punigluconin (2,3-di-O-galloyl-4, 6-(S)-hexahydroxydiphenoylgluconic acid); and
Pedunculagin (2,3,4,6-bis-(S)-hexahydroxydiphenoyl-D-glucose).

Suitable extracts may contain other low molecular weight tannins. For example, suitable extracts of Phyllanthus emblica are set forth in U.S. Pat. No. 6,124,268, Ghosal, issued Sep. 26, 2000 entitled “Natural Oxidant Compositions, Method For Obtaining Same And Cosmetic, Pharmaceutical and Nutritional Formulations Thereof” and U.S. Pat. No. 6,235,721, issued May 22, 2001. These extracts are said to comprise a blend of antioxidants referred to as “CAPROS” which contains:
about 35-55wt % of Emblicanin A /Emblicanin B (gallic/ellagic acid derivatives of 2-keto-glucono-.delta.-lactone);
about 4-15% of Punigluconin (2,3-di-O-galloyl-4, 6-(S)-hexahydroxydiphenoylgluconic acid);
about 10-20% of Pedunculagin (2,3,4,6-bis-(S)-hexahydroxydiphenoyl-D-glucose);
about 5-15% of Rutin (3′, 4′, 5,7-tetrahydroxyflavone-3-O-rhamnoglucoside); and
about 10-30% of tannoids of gallic/ellagic acid.

A particularly preferred form of the extract of Phyllanthus emblica is the extract described in U.S. Pat. No. 6,649,150 B2 issued Nov. 18, 2003, which comprises a modification of the blend of antioxidants (CAPROS) containing low levels (<1%, w/w) of total flavonoids. The total flavonoids are maintained at a level generally less than about 1.0 wt %, preferably less than about 0.8 wt %, and even more preferably less than about 0.6 wt %. with even lower contents of RUTIN. The desired concentrations of the Rutin species of flavonoids (3′,4′,5′,7-tetrahydroxyflavone-3-0-rhamnoglucoside) in the standardized extract are less than 1.0%, less than 0.01%, less than 0.001% and less than 0.0001%, with a value of 0.01 to 0.001% being particularly preferred.

Examples of such compositions can be defined as follows:

| | | | MOST |
|---|---|---|---|
| PRODUCT | % BY | PREFERRED | PREFERRED |
| IDENTITY | WEIGHT | DEVIATION | DEVIATION |
| Emblicanin A | 20-35 | +−10% | +−5% |
| Emblicanin B | 10-20 | +−10% | +−5% |
| Pedunculagin | 15-30 | +−10% | +−5% |
| Punigluconin | 3-12 | +−10% | +−5% |
| Total Flavonoids | <1 | +−10% | +−5% |

Whereas, light colored EMBLICA consists essentially of the desired components for the purposes of skin lightening or skin whitening as described above, it has been observed that black specks having a particle size of on the order of about 20 microns down to 1 micron and water-insoluble oligomeric/polymeric materials in the commercial product can diminish the esthetic appearance of the final formulations. Therefore, preferred extracts of Phyllanthus Emblica comprise, for example, less than 5% by weight of polymeric tannins, with substantially no black specks and at high levels, e.g. over 75% by weight of bio-active, low molecular-weight hydrolysable tannins having molecular weights below 1,000. The resultant extracts can be used for all applications previously described in the prior art: e.g. in cosmetic formulations, for example, skin lightening or even-toning, anti-aging and sunscreens.

Preferred concentrations of the components on a percent by weight basis of the total dried extract are : Emblicanin A 20-35% Emblicanin B 10-20% Pedunculagin 15-30% Punigluconin 3-12% Total Flavonoids<1, with the average percentage deviations from these preferred values of: Emblicanin A +/−10% to +/−5%, Emblicanin B +/−10% to +1-5%, Pedunculagin +/−10% to +/'%, Punigluconin, +−10% +−5%, total flavonoids +10% to +5%.

A suitable extract of Phyllanthus Emblica can be obtained by merely pressing the fruit or by alcoholic maceration to form a dilute-alcoholic extract, as described in French patent 2730408, published Aug. 14, 1996. Extracts such as these may be concentrated at a moderate temperature under reduced pressure, preferably less than 50° C., then optionally brought to the dry state by freeze-drying or any other method under reduced pressure and at a temperature that is lower than 50° C. so as to avoid degrading the active ingredients of the fruit. Such techniques are well known in the art and are described in detail in Examples 3, 6 and 8 of the French Patent No. 2730408.

In U.S. Pat. Nos. 6,124,268 and 6,235,721, suitable extracts of Phyllanthus emblica comprising a blend of antioxidants referred to above as “CAPROS” are obtained from the fresh fruit at elevated temperatures; e.g. 70° C., using a very dilute aqueous or alcoholic-water salt solution, e.g. 0.1 to 5%, preferably 1 to 2%. By extraction in the presence of sodium chloride, potassium chloride, calcium chloride or magnesium chloride solution, hydrolysis of the glycocidic enzymes in the plant is prevented, degradation of the anti-oxidant compounds therein by enzymes present in the fruits of the Emblica officinalis plant is prevented and the product is protected from microbial infestation. Alternatively, the anti-oxidant product is isolated using a buffer solution, e.g. 0.1% to 5% (w/w), preferably 1% to 2%, of sodium citrate/citric acid, sodium acetate/acetic acid, sodium phosphate/phosphoric acid, instead of an aqueous or alcoholic water salt solution.

The modified blends of antioxidants (CAPROS) described in U.S. Pat. No. 6,649,150 B2 containing low levels (<1%, w/w) of total flavonoids, can be obtained by removal of the total flavonoids by reversed-phase column chromatography or HPLC using a solvent system of acetonitrile, water/phosphoric acid (20/80/1) or other solvent combinations as they elute faster than the low molecular-weight tannins.

Also, by selection of geographical location, the Phyllanthus emblica fruit extract may provide a substantially lower level of the total flavonoids (<1.0%). It has been observed that medium-sized fruits collected from some parts of eastern India, during October-November, after water extraction and drying, yielded an antioxidant composition as a powder with a low level (<1.0%) of total flavonoids.

In the context of the present invention “flavonoids” include a family of compounds which exhibit a peak at 350 nm when analyzed by UV spectral data. Examples of flavonoids include but are not limited to flavonols and flavones, a species thereof being Rutin as discussed above.

Suitable Phyllanthus emblica extracts are available commercially as a powder in packaged form, e.g. in drums, in amounts of generally at least 500 g, with samples weighing about 50 g. Larger or smaller commercial shipments are also possible. The powder in the package can be analyzed to determine if it conforms to the desired specifications. In order to obtain a packaged powder with the desired specifications, different batches of powdered extract can be blended.

It has been reported that the black specks are substantially, if not completely water-insoluble as measured at room temperature, (20-25° C.) and comprise oligomeric/polymeric tannoids having no aromatic hydrogen. By water-insoluble it is meant that a 1% by weight concentration of polymeric tannin in water does not exhibit a solubility of more that 10% by weight of the total tannin at 22° C. If necessary, the black specks and/or precursors thereof and/or polymeric tannins can be separated from the water-soluble components, for example by filtration with the use of a filter aid. It is also contemplated that still other separation processes can be employed such as, for example, sedimentation, flotation and elutriation. A filter aid, e.g. diatomite filter aids, cross-linked polyvinyl pyrrolidone as well as silica and silicate sorbents can also be used to remove the oligomeric/polymeric materials. Some of the suppliers of these filter aids are Advanced Minerals (Celpure 25, 65 & 100, AW Cellite NF, MP Harborlite), International Specialty Products (Plasdone XL), United Perlite Corporation (Ultralite Perlite 505, 606C, 606F, 808, 909C, 909F). Likewise, extraction of the black specks or precursors thereof with a substantially water-miscible solvent, e.g. (ethanol, methanol, isopropanol or mixture of solvents) is also contemplated. For further details of separation systems, reference is made to descriptions in the patent and chemical engineering literature, for example, section 19 (liquid-solid systems) in Perry's Chemical Engineer's Handbook, 6th edition, editors Perry, Green and Maloney, 1984, McGraw-Hill Book Company.

The powdered Phyllanthus emblica extract is preferably incorporated in a cosmetically or pharmaceutically acceptable carrier, preferably having a pH of about between 3 to 6.5. The carrier is any conventional carrier for topical administration and is preferably employed in a concentration of about 90% to 99.7%, preferably 95 to 99.5. (In other words, the concentration of the antioxidant composition of the present invention is generally about 0.3 to 10% by weight, preferably 0.5 to 5% by weight.)

In order to attain certain objectives, formulations are provided which are not based on water, but instead are based on a substantially anhydrous or non-aqueous vehicle so that the final formulation contains preferably less than 1% by weight of water. Aside from being non-aqueous, the vehicle must be capable of dispersing the extracts with adjuvants if necessary. It is also preferred that the non-aqueous vehicle have an emollient fimction as well. Classes of vehicles to be used in the present invention include but are not limited to silicone fluids, organic esters and glycols.

Examples of silicone vehicles include but are not limited to cyclomethicone, both the tetramer and the pentamer, hexamethyldisiloxane, phenyltrimethicone cross linked polymers of dimethicone and cyclomethicone (hereinafter “crosspolymer”) methylvinylsiloxanes, methylvinylsiloxane-dimethylsiloxane copolymers, dimethylvinylsiloxy-terminated dimethylpolysiloxanes, dimethylvinylsiloxy-terminated dimethylsiloxane-methylphenylsiloxane copolymers, dimethylvinylsiloxy-terminated dimethylsiloxane-diphenylsiloxane-methylvinylsiloxane copolymers, trimethylsiloxy-terminated dimethylsiloxane-methylbinylsiloxane copolymers, trimethylsiloxy-terminated dimethylsiloxane-methylphenylsiloxane-methylbinylsiloxane copolymers, dimethylvinylsiloxy-terminated methyl(3,3,3-triflurorpropyl)polysiloxanes, and dimethylvinylsiloxy-terminated dimethylsiloxane-methyl(3,3-trifluoropropyl)siloxane copolymers, as well as functional derivatives thereof.

There are, moreover, innumerable polyorganosiloxane oils that are described in the commercial and patent literature, and it is expected that still other silicone oils will be developed in the future; so it is contemplated that all silicone oils will be useful in the present invention.

Also, U.S. Pat. No. 6,475,500 to Vatter et al. is of interest since it describes different anhydrous skin treatments including a cross linked siloxane elastomer gel of a specific yield point and volatile siloxane inclusions of the elastomers.

As for the anhydrous organic esters that can be used as vehicles in the present invention, preferred are those which also have emollient properties. Examples of such esters include but are not limited to cetearyl octanoate, caprylic/capric triglyceride, octylhydroxysterate, PPG-2 myristyl ether propionate, tentaerythrityl tetracaprylate/caprate, tentaerythrityl tetraisosterate, natural and synthetic jojoba oils, cetyl acetate, and acetylated lanolin alcohol.

Examples of glycols, include but are not limited to mono- or poly-alkylene glycols are contemplated, a non-limiting example being propylene glycol.

In the substantially anhydrous formulation, the content of the vehicle is sufficient, generally, is about 20-80%, preferably 20-60% by weight of the completed formulation to achieve the desired dispersibility of the extracts. The content of extracts in the formulation is generally about from 0.05 to 10%, preferably 0.1-3% by weight, with the preferred minimum weight ratio of the content of the vehicle to the content of the extracts being about 20:3.

Structural/gelling agents can be combined with the Phyllanthus emblica extracts to form a mixture comprising the extract with the structural agent, and/or the gelling agent. Likewise, the structural/gelling agent can be combined with the substantially anhydrous vehicle in order to form corresponding mixtures which thereafter can be combined with the extracts.

The structural agent provides firmness, structure, consistency and thermal stability to the product that can be selected from subgeneric classes of materials which include but are not limited to natural, modified or unmodified waxes, mineral waxes, high melting point fatty alcohols, glycerol or glycol esters, polyethylene and polyethylene glycol polymers.

Examples of the natural modified or unmodified waxes include but are not limited to beeswax, candelilla wax, carnauba wax, and hydrogenated castor wax.

Examples of mineral waxes include but are not limited to ozokerite and ceresin.

Examples of high melting fatty alcohols include but are not limited to cetyl alcohol and stearyl alcohol.

Examples of glycerol or glycol esters include but are not limited to Croda Syncrowaxes, i.e. 18-36 glycol esters.

An example of polyethylene glycol polymers includes but is not limited to Carbowax Sentry 1000.

The structural agents are incorporated in the final formulation at a level of about 5-50% by weight.

As for gelling agents which are also used in an amount of 5-50% by weight of the final formulation, subgeneric classes include but are not limited to silicone elastomers, gelled natural and mineral oil systems and gelled mineral oil and polymer systems.

Preferred examples of silicone elastomers include but are not limited to cyclomethicone and dimethicone cross polymers e.g. Dow Corning 9040, polysilicone-11 mixtures, e.g. Gransil PM Gel, and Gransil DCM, and Gransil DMG-6.

Preferred examples of gelled natural and mineral oil systems include but are not limited to a mixture of canola oil and silica and corn starch, e.g. Vegelatum Clear; a mixture of canola oil, soy bean germ extract, corn starch and silica, e.g. Vegelatum Equiline; gelled castor oil and rice bran oil (Natunola Health).

Preferred examples of gelled mineral oil and polymer systems include but are not limited to esters of hydrogenated polyisobutene, ethylene/propylene/styrene copolymers, and butylene/ethylene/styrene copolymers, e.g. Versagel M, ME, MC, MD, ME, MJ and MP (Penreco Corp.); and polybutene, e.g. Indopol H-100.

The substantially anhydrous delivery system can be utilized for the incorporation of any -extract; however, the delivery system is particularly beneficial for the incorporation of the standardized extract described above and especially the commercial product. It is also contemplated that the anhydrous delivery system of the present invention can be utilized for the incorporation of other active materials.

To summarize the quantitative amounts described above, the final formulation on a weight basis comprises in general about 20-80%, preferably 20-60% of a substantially anhydrous liquid vehicle, a total of about 5 to about 90% of a structural and/or gelling agent, and 0.05-10%, preferably 0.1-3% of a extract, especially the extract having the trademark EMBLICA.

Based on 100 parts by weight of the fmal formulation, the first step comprises blending a mixture of about 5 to 80% of a vehicle and 5 to 90 of a structural or gelling agent with sufficient heat, e.g. a temperature of about 60 to 90° C. and mixing until a clear and uniform mixture is obtained.

It is contemplated that the present invention embraces any form of Phyllanthus emblica extract which will lead to a synergistic response as defined herein. The extract need not provide this function for embodiments which do not require a synergistic response as defined herein. Further examples being the topical anhydrous delivery system described above and in U.S. Patent Publication 20040076699 and the enriched aqueous composition of emblica officinalis described in U.S. Patent Publication 20040126446.

The licorice extracts employed in the present invention are well known skin lightening agents and contain about 20% of glabridin, this extract also known as Glycerrhiza glabra. It is contemplated that the extracts can be formulated so that they constitute more or less than 20% glabridin from a very weak amount to a highly concentrated amount, for example from 1% to 100%. Where required, the concentration of glabridin is such that the use of the licorice extract leads to a synergistic response in changing skin tone or stretch mark reduction or repairing photodamaged skin when used together with the extract of phyllanthus emblica and niacinamide, as compared to an equal amount of any of these three components individually.

Niacinamide, the amide of 3-pyridine carboxylic acid, is also a known skin lightening agent.

The ratios of the three active components with respect to one another which provide a synergistic response in changing skin tone or stretch mark reduction or repairing photodamaged skin or photoprotecting skin as compared to an equal amount of any of the three individual active components on a weight basis, include the prescribed ratios described above. These ratios include:
the ratio of the extract of Phyllanthus emblica to the extract of licorice of about 2 to 0.01, preferably 1 to 0.05 and even more preferably 1 to 0.1;
the ratio of phyllanthus emblica to niacinamide of from 0.5 to 10, preferably 1 to 8, particularly 1 to 5; and
the ratio of the extract of licorice to the niacinamide of about 0.01 to 10, preferably 0.05 to 5, and more preferably 0.1 to 5, all on a weight basis of dry solids.

In preferred embodiments, the ratios that provide a synergistic response are as follows:
the ratio of the extract of Phyllanthus emblica to the extract of licorice is from 1 to 0.05 and even more preferably 1 to 0.1;
the ratio of phyllanthus emblica to niacinamide is from 1 to 8, and even more preferably 1 to 5; and
the ratio of the extract of licorice to the niacinamide is from 0.05 to 5, and more preferably 0.1 to 5; all on a weight basis of dry solids.

For the even more preferred embodiments, the ratios that provide a synergistic response are as follows:
the ratio of the extract of Phyllanthus emblica to the extract of licorice is from 1 to 0.1;
the ratio of phyllanthus emblica to niacinamide is from 1 to 5; and
the ratio of the extract of licorice to the niacinamide is 0.1 to 5, all on a weight basis of dry solids.

In addition to cosmetic skin lightening, the skin care compositions of this invention can be used to treat hyper pigmented skin, the types of hyper pigmentation being (a) genetic/environmental: (freckles=ephelides, xeroderma pigmentosum), (b)disease-related, (c) pregnancy-related, (d) enzymatic deficiencies and (e) uv-induced.

Freckles are areas where the melanocytes are more active and more responsive to UV radiation than in neighboring skin. This is mainly associated with fair-skinned people and related to the presence of red hair (genetic predisposition). The present inventive skin care compositions can also be used to reduce age spots.

The skin care compositions of the present invention can also contain one or more additional ingredients which can decrease pigmentation, including but not limited to soy milk, ascorbic acid, ascorbyl glucoside, and others known in the art such as those mentioned in Published U.S. Application 2004/0028642 A1, published Feb. 12, 2004, page 8, right hand column, paragraphs 0077 and 0078, incorporated by reference herein, as well as those mentioned in Published U.S. Application 2004/0253332 A1 published Dec. 16, 2004, paragraph 0049, these applications also being incorporated by reference herein.

Likewise, the skin care compositions of the invention can also include any of the known topical excipients suggested in the latter published application in paragraph 0050 as well as in U.S. Pat. No. 6,124,268.

For photo protective applications, if sunscreens are added, there can be used any agent capable of protecting skin from UV exposure, including but not limited to those set forth in the latter published application '332 A1, paragraph 0048.

Other known ingredients include, but are not limited to antioxidants, polymers, skin penetration enhancers, anti-inflammatory agents and moisturizers.

The term penetration enhancer is applied to materials that have a direct effect on the permeability of the skin barrier. Some materials may act by a direct chemical insult on the skin while others may not have a specific barrier effect. The latter may affect the solubility and/or dispersibility of the medicament and/or its delivery system (the vehicle). A variety of organic penetration enhancer include dimethyl sulfoxide, isopropyl myristate, decyl, undecyl or dodecyl alcohol, propylene glycol, polyethylene glycol or 9-11C, 12-13C or 12-15C fatty alcohols, azone, alkyl pyrrolidones, lecithin etc. Surfactants can also be used as penetration enhancers. The effect of surfactants as penetration enhancers has been attributed to their ability to bind protein, thereby altering the structure of the stratum comeum.

The resultant composition and excipients are administered topically and preferably are incorporated in a topical cosmetically acceptable carrier, the concentration of the carrier being adjustable over a wide range e.g. from about 60% to 99.9% by weight. The concentration of the total on a dry weight basis of the extract of phyllanthus emblica, licorice extracts and niacinamide can also be varied widely, generally from about 0.1 to 40% by weight, preferably 0.3 to 10% by weight, more preferably 0.5 to 5% by weight.

When used to achieve skin lightening or reducing a stretch mark or repairing photodamaged skin or photoprotecting skin, the amount, duration and frequency at which the skin care composition is applied to the skin should be sufficient to visibly change or improve the appearance of the skin. For example, the skin care composition can be used for at least once per day for a period of preferably at least about 2 weeks. The volume of the skin care composition applied to the skin can vary widely. Amounts within the range of 0.5-5.0 ml/m2 of skin are suitable. Preferably, the volume of the skin care. composition applied to the skin is consistent with commercial skin care lotions.

When used as a sunscreen composition and the like, the amount, duration and frequency at which the skin care composition is to be applied to the skin is generally in accordance with the recommended instructions for commercial sunscreen compositions, e.g. apply about 30 minutes prior to exposure to the sun and the thicker the application the greater the protection.

When used as a composition for photoprotecting skin, the amount, duration and frequency at which the skin care composition is to be applied to the skin is generally in accordance with the recommended instructions for commercial sunscreens or compositions for photoprotecting skin, e.g. apply about 30 minutes prior to exposure to the sun and the thicker the application the greater the protection.

When used as a composition for repairing photo-damaged skin, the amount, duration and frequency at which the skin care composition is to be applied to the skin is generally in accordance with the recommended instructions for commercial compositions for repairing photo-damaged skin, e.g. apply once a day or in the evening as needed and avoid further exposure to the sun.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

EXAMPLE 1
Skin Lightening Cream

| INCI name | Trade Name/Supplier | % w/w |
|---|---|---|
| Phase A | | |
| Water (demineralized) | | 60.25 |
| Disodium EDTA | Triplex III/Rona | 0.05 |
| Glycerin (99.7%) | Emery 916/Cognis | 3.00 |
| Lecithin | Emulmetik 100/Lucas Meyer | 0.50 |
| Xantham gum | Vanzan NF/ Vanderbilt | 0.25 |
| Phase B | | |
| C12-15 Akyl Benzoate | Finsolv TN/Finetex | 5.00 |
| Caprylic Capric | Myritol 318/Cognis | 4.00 |
| Triglycerides | | |
| Sunflower Seed Oil | Lipovol Sun/Lipo | 3.50 |
| Bisabolol | Ronacare Bisabolol/EMD | 0.20 |
| Shea Butter | Cetiol SB-45/Cognis | 3.50 |
| Dimethicone | Dow Corning 200, 100 | 1.00 |
| | cst/Dow Corning | |
| Glyceryl Stearate, | Arlacel 165/Uniqema | 2.50 |
| PEG-100 Stearate | | |
| Glyceryl Stearate | Cerasynt SD/ISP | 1.00 |
| Sorbitan Stearate | Arlacel 60/Uniquema | 1.00 |
| Cetyl Alcohol | Crodacol C-70 | 1.00 |
| Stearic Acid | Emersol 132/Cognis | 1.80 |
| Phase C | | |
| Water (demineralized) | | 5.00 |
| Phyllanthus emblica | Emblica ™/EMD | 1.00 |
| fruit Extract | | |
| Niacinamide | RonaCare ™ Niacin/EMD | 3.00 |
| Phase D | | |
| Butylene Glycol | | 2.00 |
| Glycerrhiza glabra | Licorice PTH/Barnet | 0.10 |
| root extract | | |
| Phase D | | |
| Polyacrylamide, | Sepigel 305/Seppic | 1.00 |
| C13-14 Isoparaffin, | | |
| Laureth-7 | | |
| Phase E | | |
| Triethanolamine | | 0.40 |
| Phase F | | |
| DMDM Hydantoin, | Paragon II/McIntyre | 1.00 |
| Propylparaben, | | |
| Methylparaben | | |
| Total | | 100.00 |

Procedure: Disperse Xanthan Gum in water while stirring and heat A to 75° C.. Combine ingredients of phase B, then heat to 75° C.. Add phase B to A with good mixing. Homogenize mixture with moderate speed, while cooling to 50° C.. Stop homogenizing when temperature reaches 40° C.. Add the premixed phase C & D; stir gently until mixture is homogeneous. Adjust pH with phase F to 4.5 to 5.0, then add phase E & D.
Notes
pH value: −4.8
Viscosity: 24,000 cps (Brookfield RVT, spindle C, Helipath) at 25° C..

EXAMPLE 2
Sunscreen Lotion
Formulation # EUS15-99

| | Trade Name/ | |
|---|---|---|
| INCI name | Manufacturer | % w/w |
| Phase A-1 | | |
| Water (demineralized) | | 66.42 |
| Disodium EDTA | | 0.05 |
| Phase A-2 | | |
| Acrylated/C10-30 Alkyl | | 0.05 |
| Acrylates Crosspolymer | | |
| Phase B | | |
| Octyl Methoxycinnamate | Eusolex 2292/RONA | 7.50 |
| Homosalate | Eusolex HMS/RONA | 5.00 |
| Isopropyl Myristate | Emerest 2314/Cognis | 4.00 |
| C12-15 Alkyl Benzoate | Finsolv TN/Finetex | 4.00 |
| Cetyl alcohol | Crodacol C-70/Croda | 1.50 |
| Stereath-2 | Brij 72/Uniqema | 2.00 |
| Steareth-21 | Brij 721/Uniqema | 2.50 |
| Dimethicone | Dow Corning 200 Fluid, | 0.50 |
| | 100 cst/Dow Corning | |
| Glycerrhiza glabra | Present invention | 0.10% |
| Phase C | | |
| Triethanolamine | TEA 99%/Union Carbide | 0.23 |
| Phase D | | |
| Water (demineralized) | | 5.00 |
| Phyllanthus emblica | Emblica ™/EMD | 0.50 |
| fruit extract | | |
| Niacinamide | Present invention | 2.00 |
| Phase E | | |
| Phenoxyethanol, | Liquapar PE/Sutton | 1.00 |
| Isopropylparaben, | | |
| Isobutylparaben, | | |
| Butylparaben | | |
| Total | | 100.00 |

Procedure: Disperse A-2 in A-1. Heat A to 70-75° C.. Combine Phase B and heat to 70-75° C.. Add phase B to Phase A while stirring. Add Phase C.. Homogenize until mixture cools to 45-40° C. At 30° C.. add Phase D. Mix until uniform. Add Phase E. Stir allowing mixture to cool to RT.

Broad Spectrum Sunscreen Lotions

| | EXAMPLE 3 | EXAMPLE 4 | EXAMPLE 5 |
|---|---|---|---|
| | 8A SPF 30 | 8B SPF 40 | 8C SPF 45 |
| INCI name | % w/w | % w/w | % w/w |
| Phase A-1 | | | |
| Deionized water | qs | qs | qs |
| Disodium EDTA | 0.05 | 0.05 | 0.05 |
| Propylene Glycol | 3.00 | 3.00 | 3.00 |
| Glycerin | 2.00 | 2.00 | 2.00 |
| Phase A-2 | | | |
| Xanthan Gum | 0.10 | 0.10 | 0.10 |
| Acrylates/C10-30 | 0.08 | 0.08 | 0.08 |
| Alkyl Acrylate | | | |
| Copolymer | | | |
| Phase B | | | |
| Cetyl Alcohol, | 3.50 | 3.50 | 3.50 |
| Glyceryl Stearate, | | | |
| PEG-75, Ceteth-20 | | | |
| and Steareth-20 | | | |
| Dimethicone | 0.50 | 0.50 | 0.50 |
| C30-38 Olefin/ | 1.00 | 1.00 | 1.00 |
| Isopropyl Maleate/ | | | |
| MA Copolymer | | | |
| C12-15 Alkyl | 6.00 | 6.00 | 6.00 |
| Benzoate | | | |
| Butyl Methoxydi- | 2.00 | 2.00 | 2.00 |
| benzoymethane | | | |
| Homosalate | 15.00 | 15.00 | 15.00 |
| Octisalate | 5.00 | 5.00 | 5.00 |
| Diethylhexyl | 0.00 | 2.00 | 3.00 |
| Syringylidene | | | |
| Malonate (Oxynex ST) | | | |
| Phase C | | | |
| Water | 5.00 | 5.00 | 5.00 |
| (demineralized) | | | |
| Phyllanthus emblica | 1.00 | 1.00 | 1.00 |
| fruit extract | | | |
| Niacinamide | 3.00 | 3.00 | 3.00 |
| Phase D | | | |
| Butylene glycol | 2.00 | 2.00 | 2.00 |
| Glycerrhiza glabra | 0.10 | 0.10 | 0.10 |
| root extract | | | |
| Phase E | | | |
| Triethanolamine (99%) | 0.15 | 0.15 | 0.15 |
| Phase F | | | |
| Phenoxyethanol (and) | 1.00 | 1.00 | 1.00 |
| Isopropylparaben | | | |
| (and) Isobutyl- | | | |
| paraben (and) | | | |
| Butylparaben | 100.00 | 100.00 | 100.00 |
| Total | 100.00 | 100.00 | 100.00 |

Procedure: Combine ingredients of phase A-1. Disperse A-1 in A-2 with continuous agitation, then heat to 75° C.. Combine ingredients of phase B and heat to 75-80° C.. Add phase B to A under mixing conditions. Homogenize for 5-10 min.; add phase C required for neutralization. At 40° C. add phase D. Mix until uniform.

Skin Lightening Study Results
Methodology
Human volunteers—Asians and Hispanics
   Skin type III (burns moderately/tans gradually) and IV (burns minimally/always tans well); Defined by Fitzpatrick phototype scale
Study duration
   9 weeks
Test sites
   Upper arms, left & right
Test substances
   Study 1: 1% Emblica vs 0.1% Licorice (Asians, 15 subjects),
   Study 2: 2% Emblica vs 2% Emblica+0.1% Licorice extract+3% Niacinamide (Hispanics, 16 subjects).
Application frequency
   0.5 ml twice a day
Results
   Represented using the individual typology angle (COLIPA SFP test method); Measured by chromometric measurement; Difference in ITA° (ΔE) was calculated by subtracting average ITA° of the treated site from that of the average baseline (first day of the study).

[image]

### BRIEF DESCRIPTION OF THE GRAPHS

Graph 1 shows the results of skin lightening study 1 and 2;

Graph 2 shows the calculation of various “Individual topology angles,” the parameter used to define skin tone.

Method of Calculation of ITA°
ITA°=[Arc Tangent((L*−50)/b*]180/3.1416
L* value—Lightness
a* value—color in red-green axis
b* value—Color in blue-yellow axis
ITA° Skin Color
>55 Very Light
41 to 55 Light
28 to 41 Interrnediate
10 to 28 Tan
−30 to 10 Brown
<−30 Black

[image]

### Comparative Tyrosinase Inhibitory Activity

Methodology

Experimental Conditions

All measurements were performed at room temperature (24 C) with a Beckman Coulter DU-640 spectrophotometer at a scan speed of 1200 nm/min with a spectral range from 300 to 700 nm.

Preparation of Stock Solutions

a) Stock Solutions

1. Buffer solution
   KH₂PO₄ 99.8% & KOH 85+% (pH 7.4, 100 mM)
   Mol. Wt. 136.09, VWR and Sigma-Aldrich
   KH₂PO₄ (1362 mg) dissolved in distilled water (100 ml) and pH adjusted to 7.4 with a 2 M KOH solution
2. Mushroom Tyrosinase solution (240 units per 1 ml)
   2440 units/mg, Sigma-Aldrich
   Tyrosinase (2.5 mg) dissolved in 25 ml of distilled water
3. L-DOPA solution (6.3 mM)
   Mol. Wt. 197, Sigma-Aldrich
   33.1 mg are dissolved in distilled water (25 ml)
4. L-Tyrosine solution (1.3 mM)
   Mol. Wt. 181.2, Sigma-Aldrich
   23.5 mg are dissolved in distilled water (100 ml)
5. Hydrogen peroxide solution (5 mM)
   Sigma-Aldrich
   1/100 of 0.9/25 vol. dilution of a 50% wt. solution
      b) Skin Lightening Ingredients
5. Emblica™ antioxidant (5 mM)—Lot # CA0106007
   Average Mol. Wt. 729, EMD Chemicals / Merck KGaA
   Emblica (18.2 mg) dissolved in distilled water (5 ml)
6. Kojic acid (26.7 mM)
   Mol. Wt. 142, Sigma-Aldrich
   Kojic acid (19 mg) dissolved in distilled water (5 ml)
7. Hydroquinone (40 mM)
   Mol. Wt. 110, Eastman
   Hydroquinone (22 mg) dissolved in distilled water (5 ml)
8. Licorice extract—PTH (glabridin content˜20%)—Lot # 60309262 (*)
   Barnet Products
   Licorice extract (22.9 mg) dissolved in denatured ethanol (5 ml)
      * Values have been corrected by the respective alcoholic/aqueous solutions as they influence the conversion of L-DOPA and L-Tyrosine into dopachrome by tyrosinase.
      Procedure

The stock solutions were added in an orderly manner with quantities according to Table 1 as to obtain a final total volume of 3.0 ml for each preparation. First, the substrate (L-DOPA or L-Tyrosine) was mixed with the hydrogen peroxide and the buffer solutions. Proportions are different due to solubility reasons and details are given in Tables la and lb. This mixture was incubated for 10 min at 25C. Then, 2 ml of this mixture was added to 1 ml of mixture solution containing tyrosinase, ingredient X and buffer solution according to Table 1. After 15 min of reaction time, the spectra were recorded between 300 and 700 nm and the optical density at 475 nm (Dopachrome) measured.

TABLE 1a
L-DOPA substrate

| | | HYDROGEN | | | | |
|---|---|---|---|---|---|---|
| | L-DOPA | PEROXIDE | BUFFER STOCK | PEROXIDASE | INGREDIENT | BUFFER |
| PREPARATION | SOLUTION | SOLUTION | SOLUTION | SOLUTION | X SOLUTION | SOLUTION |
| A | 0.5 ml | 0.5 ml | 1.0 ml | 0.5 ml | 0 ml | 0.5 ml |
| B | 0.5 ml | 0.5 ml | 1.0 ml | 0.5 ml | 0.1 ml | 0.4 ml |
| C | 0.5 ml | 0.5 ml | 1.0 ml | 0.5 ml | 0.2 ml | 0.3 ml |
| D | 0.5 ml | 0.5 ml | 1.0 ml | 0.5 ml | 0.3 ml | 0.2 ml |
| E | 0.5 ml | 0.5 ml | 1.0 ml | 0.5 ml | 0.4 ml | 0.1 ml |

TABLE 1b
L-Tyrosine substrate

| | | HYDROGEN | | | | |
|---|---|---|---|---|---|---|
| | L-TYROSINE | PEROXIDE | BUFFER STOCK | PEROXIDASE | INGREDIENT | BUFFER |
| PREPARATION | SOLUTION | SOLUTION | SOLUTION | SOLUTION | X SOLUTION | SOLUTION |
| A | 1.0 ml | 0.5 ml | 0.5 ml | 0.5 ml | 0 ml | 0.5 ml |
| B | 1.0 ml | 0.5 ml | 0.5 ml | 0.5 ml | 0.1 ml | 0.4 ml |
| C | 1.0 ml | 0.5 ml | 0.5 ml | 0.5 ml | 0.2 ml | 0.3 ml |
| D | 1.0 ml | 0.5 ml | 0.5 ml | 0.5 ml | 0.3 ml | 0.2 ml |
| E | 1.0 ml | 0.5 ml | 0.5 ml | 0.5 ml | 0.4 ml | 0.1 ml |

Results and Discussion

Results are shown in Table 2. The results demonstrate that Emblica™ has a good inhibitory activity against tyrosinase using both substrates (DOPA and Tyrosine). Licorice extract showed excellent inhibitory activity against both substrates. Kojic acid shows good inhibitory activity as well. Hydroquinone showed excellent inhibitory activity when tyrosine was used as a substrate, but less effective with DOPA.

TABLE 2

Tyrosinase inhibition
IC_{50%} (μg/ml)

| Compound | L-DOPA substrate | Tyrosine substrate |
|---|---|---|
| Licorice extract | 12 | 4 |
| Kojic acid | 35 | 4 |
| Ascorbic acid | 60 | 32 |
| Emblica | 135 | 64 |
| Hydroquinone | 233 | 1.5 |

The preceding examples can be repeated with similar success by substituting the generically or specifically described reactants and/or operating conditions of this invention for those used in the preceding examples.

The entire disclosures of all applications, patents and publications, cited herein and of corresponding U.S. Provisional Application Ser. No. 60/696,788, filed Jul. 7, 2005, are incorporated by reference herein.

From the foregoing description, one skilled in the art can easily ascertain the essential characteristics of this invention and, without departing from the spirit and scope thereof, can make various changes and modifications of the invention to adapt it to various usages and conditions.

1. A skin care composition comprising an extract of Phyllanthus emblica, a licorice extract, and niacinamide in proportions which provide a synergistic response in changing skin tone as compared to an equal amount of any of the individual components, on a weight basis.

2. A skin care composition as in claim 1 additionally comprising a pharmaceutically or cosmetically acceptable carrier(s).

3. A skin care composition according to claim 2, wherein said extract of phyllanthus emblica is any of standardized compositions according to the claims of U.S. Pat. No. 6,649,150.

4. A skin care composition according to claim 3, wherein said extract of licorice comprises about 20% by weight of glabridin.

5. A skin care composition according to claim 2 which is a skin lightening/even-toning composition, a sunscreen composition, a composition for photoprotecting skin or a composition for repairing photo-damaged skin.

6. A skin care composition according to claim 4 which is a skin lightening/even-toning composition.

7. A skin care composition according to claim 4 which is a sunscreen composition.

8. A skin care composition according to claim 4 which is a composition for photoprotecting skin.

9. A skin care composition according to claim 4 which is a composition for repairing photo-damaged skin.

10. A skin care composition according to claim 4 which is a composition for reducing the appearance of stretch marks.

11. A sunscreen composition comprising a sunscreen agent and/or anti-inflammatory agent and an extract of Phyllanthus emblica, a licorice extract, and niacinamide in proportions which provide a synergistic response in changing skin tone as compared to an equal amount of any of the individual components, on a weight basis.

12. A sunscreen composition as in claim 11 additionally comprising a pharmaceutically or cosmetically acceptable carrier(s).

13. A sunscreen composition according to claim 11, wherein said extract of Phyllanthus emblica is any of standardized compositions according to the claims of U.S. Pat. No. 6,649,150.

14. A sunscreen composition according to claim 13, wherein said extract of licorice comprises about 20% by weight of glabridin.

15. A composition for repairing photo-damaged skin according to claim 9 additionally comprising one or more of a UV-A sunscreen, a UV-B sunscreen, an antioxidant, a photostabilizer an anti-inflammatory agent, a moisturizer, or a combination thereof.

16. A skin care composition comprising an extract of Phyllanthus emblica, a licorice extract, and niacinamide in the following ratios on a weight basis of dry solids:
a ratio of the extract of Phyllanthus emblica to the extract of licorice of from 2 to 0.01;
a ratio of phyllanthus emblica to niacinamide of from 0.5 to 10, and
a ratio of the extract of licorice to the niacinamide of from 0.01 to 10.

17. A skin care composition as in claim 16 with the following ratios on a weight basis of dry solids:
a ratio of the extract of Phyllanthus emblica to the extract of licorice of from 1 to 0.05;
a ratio of phyllanthus emblica to niacinamide of from 1 to 8; and
a ratio of the extract of licorice to the niacinamide of from 0.05 to 5.

18. A skin care composition as in claim 16 with the following ratios on a weight basis of dry solids:
a ratio of the extract of Phyllanthus emblica to the extract of licorice of from 1 to 0.1;
a ratio of phyllanthus emblica to niacinamide of from 1 to 5; and
a ratio of the extract of licorice to the niacinamide of from 0.1 to 5.

19. A skin care composition as in claim 16 additionally comprising a pharmaceutically or cosmetically acceptable carrier(s).

20. A skin care composition according to claim 16, wherein said extract of licorice comprises about 20% by weight of glabridin.

21. A skin care composition according to claim 16 which is a skin lightening/even-toning composition, a sunscreen composition, a composition for photoprotecting skin or a composition for repairing photo-damaged skin.
